(19) United States
(12) Patent Application Publication
(10) Pub. No.: US 2024/0050985 A1
(43) Pub. Date: Feb. 15, 2024

(54) METHOD OF MANUFACTURING A HAND HEALTHCARE GLOVE

(71) Applicant: N.S. UNI-GLOVES SDN. BHD., SEREMBAN (MY)

(72) Inventors: SEE KEONG WONG, SEREMBAN (MY); PEK WEE WONG, SEREMBAN (MY)

(21) Appl. No.: 17/888,401

(22) Filed: Aug. 15, 2022

(51) Int. Cl. B05D 7/24; B05D 3/10; B05D 3/02; B05D 7/02; C08L 7/02; A61K 9/00; A61K 35/35; A61K 31/355

(52) U.S. Cl. CPC B05D 7/24; B05D 3/101; B05D 3/0254; B05D 7/02; C08L 7/02; A61K 9/0014; A61K 35/35; A61K 31/355

(57) ABSTRACT

A process for creating a disposable glove which has been specially treated with a uniform coating of the Lanolin and Vitamin-E composite through a specially controlled application and drying process. The process satisfies the need to minimize the side effects of skin damage and irritation due to prolonged and extensive application of hand gloves in the health care industry.

### BACKGROUND

This invention relates specifically to the needs of incorporating hand health-care features and benefits into gloves by forming a coating of purified Lanolin and Vitamin E onto the donning surface of disposable gloves to soothe the hands during and after a prolonged and routine application of disposable gloves.

With the increased demands and awareness from the general public for healthcare professionals to be accountable for their actions, good infection control is crucial in nursing practices. To achieve higher standards of clinical practice, safe practices to minimize the risk of cross-infection have become the uppermost in the minds of healthcare professionals when caring for patients. Disposable gloves are commonly used as a protective device for the purpose. In the process of escalating dependence for such essential protective device in today's modern health-care industry, thirty-five percent of healthcare workers who wear medical gloves may experience non-allergic skin problems at one time or another (9). The most frequently experienced is irritant contact dermatitis. The characteristics of this non-immune reaction consist of dry, crusty, hard bumps along with horizontal skin cracks. Frequent hand washing, strong surgical scrubbing agents, soaps, detergents, glove powders and the hot moist environment caused by glove wear are all associated with irritant contact dermatitis. Every time we wash our hands, we remove the natural oils that are essential for health skin. When there is a deficiency of natural oils, the skin tends to dry and loses its resilience and eventually cracks. This skin reaction may be minimized by avoiding as much as possible all of the causative factors in the workplace and home environment. In this case, the use of an emollient like lanolin has been found helpful.

The history of lanolin is both long and fascinating (1,2,3). Lanolin was known to and used by the ancient Greeks as an excellent emollient. Its strength as an active ingredient in skincare application lies in its extensive records of safe use for the purpose. Lanolin is a mixture of esters derived from several fatty acids. With water it readily forms an emulsion. The literatures on skin surface studies have provided objective evidence and confirmed the emollient effect of lanolin and its derivatives (4,5,10,14,15). Lanolin speeds up natural skin repair and also acts as an anti-irritant (13). In addition to this, lanolin has demonstrated remarkable chemical and physical similarities in comparison to human skin lipids (6,11). Its presence will help to condition the skin of user's hands.

The invention to apply Lanolin onto gloves is further enriched by the addition of Vitamin E into the formulation. Vitamin E is a powerful biological antioxidant (7,8,12) that serves to prevent disease and premature aging. It has a neutralizing effect on free radicals, a by-product of energy metabolism. Vitamin E is an important antioxidant commonly known for its ability to aid in the healing of previously damaged skin. It also tends to improve skin elasticity and thus promotes a youthful and healthy look to the skin.

The application of the moisturizing and therapeutic ingredients made of emulsified Lanolin and Vitamin E (known as Lano-E composite herein) to the gloves will substantially provide additional hand health care features, in addition to, its basic function as a barrier to control cross contamination, especially in the medical and laboratory procedures.

Disadvantages of the conventional gloves are such that offline Lano-E treatment process is labor intensive as it requires manpower to do manual glove inversion as well as operate rotating drum and drying machine for chlorination, rinsing, Lano-E treatment and drying process as shown in FIG. 3. The gloves are also need to be packed offline by labors after the offline Lano-E treatment process.

Another conventional disadvantage is that the process also consumes electricity, gas and water more than online treatment process. In addition, offline treatment process requires further post treatment process to treat waste water. The lead time for the whole process from glove manufacturing until offline treatment process including transition period is longer.

### SUMMARY OF THE INVENTION

Therefore, the present invention is contrived to overcome the conventional disadvantages. An objective of the present invention is to provide online Lano-E treatment process that does not require additional manpower to operate rotating drum and drying machine as they are not used in online treatment process. The process only requires automatic spraying nozzles as explained in detailed description part of the preferred embodiment. Online treatment process has automatic robotic grippers at stripping area to invert out the gloves to their normal orientation at stage 1 in FIG. 5 unlike offline treatment process which requires manual glove inversion. The gloves can also be directly packed online after stripping process using online packing machines.

Since the coated gloves are inverted automatically by robotic grippers, the process can help to reduce contamination by minimizing human handling at donning side of gloves. In addition, the process can also conserve energy by reducing electricity, gas and water consumption. The lead time to manufacture Lano-E coated glove via online treatment process is shorter.

The present invention satisfies the need to minimize the side effects of skin damage and irritation due to prolonged and extensive application of gloves. This invention herein describes a new disposable glove that has been specially treated with a uniform coating of Lano-E composite through a specially controlled drying process and a method of manufacturing such hand healthcare glove. The glove can be made of natural rubber latex or synthetic materials, such as, acrylonitrile butadiene, polyurethane, polychloroprene and polyvinyl chloride.

The glove that is manufactured in this manner has the following attributes during use:
a) The multiple cycles of washing and rinsing processes by chlorination remove the powder residue, kill microorganisms and remove water soluble impurities including allergenic proteins. The powder residue exists in the form of cornstarch for donning purposes that is applied in stage 18 (referring to FIG. 4) of the process and calcium carbonate as a mold release agent that is applied in stage 8 of the process. There are also powder residues of accelerators, sulphur, zinc oxide, and anti-oxidants, which are vulcanizing chemicals and preservatives that are applied in stage 10 of the process.
b) The glove possesses a coating of Lano-E composite on the donning surface that can help the hands to maintain skin elasticity and to heal previously damaged skin.
c) The Lano-E composite is formulated in such a way that the composition is adequate to provide a well-balanced, non-greasy optimum smoothness without affecting the durability and flexibility of the bulk materials of the glove.
d) The dried Lanolin and Vitamin E becomes emulsified and are released from the interior surface of the glove upon being dampened by sweat during use. The emulsified mixture is then released onto the user's hands, which helps to condition and moisturize the skin.

A method of manufacturing a hand healthcare disposable glove includes the following steps. Initially an online powdered glove is formed from natural rubber latex or other synthetic materials like acrylonitrile butadiene, polyurethane, polychloroprene or polyvinyl chloride through a dipping process. The normal orientation of the glove has an interior with a donning surface and an exterior surface. An initial step of offline treatment process is inverting the powdered glove inside out, so the donning surface is exposed. Then, subjecting the glove through a series of controlled chlorination washing processes. Coating the donning surface of the glove with a coating of an emulsified mixture of Lanolin and Vitamin E using water as a liquid medium. Evaporating the liquid medium from the emulsified mixture in a temperature controlled heating condition to achieve a dried coating of purified lanolin enriched with Vitamin E formed on the donning surface of the glove.

Lano-E online treatment process is newly added as shown in FIG. 5. This process is particularly designed for online powder free glove that does not require offline treatment process. The donning surface of glove is coated with Lano-E composite at stage 12 of the process and dried in a controlled heating condition before reaching the stripping area.

Although the present invention is briefly summarized, a fuller understanding of the invention can be obtained from the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 Shows a front view of the glove, depicting the exterior surface and the donning surface of the interior portion;

FIG. 2 Shows a cutaway view of the glove along the x-x line of FIG. 1;

FIG. 3 shows a flow chart for the application of the Lano-E coating;

FIG. 4 shows gloves manufacturing process flow outline, which is the production of the gloves prior to the process of applying the Lano-E coating; and

FIG. 5 shows an online application of Lano-E coating on gloves.

### DETAILED DESCRIPTION OF THE INVENTION

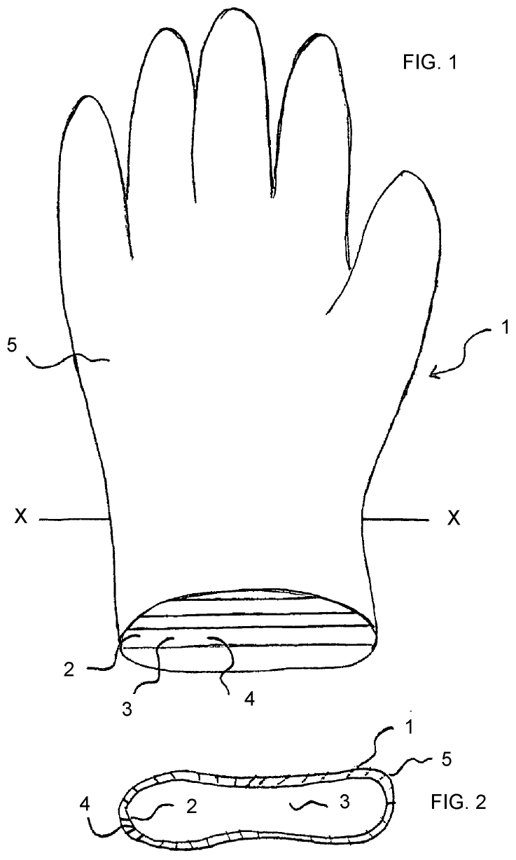

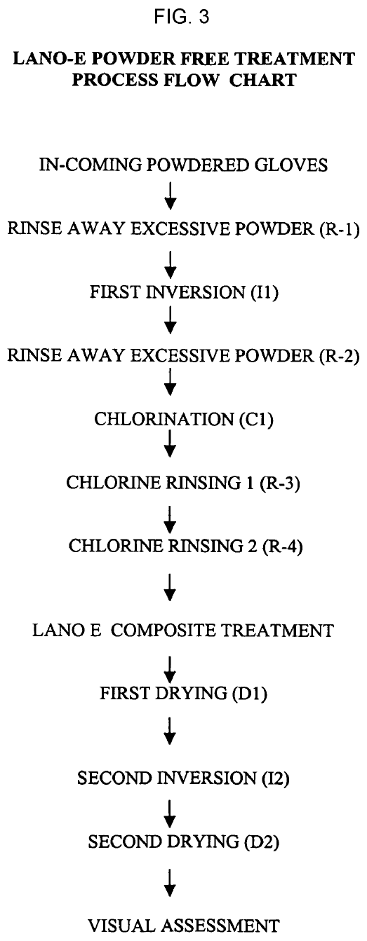

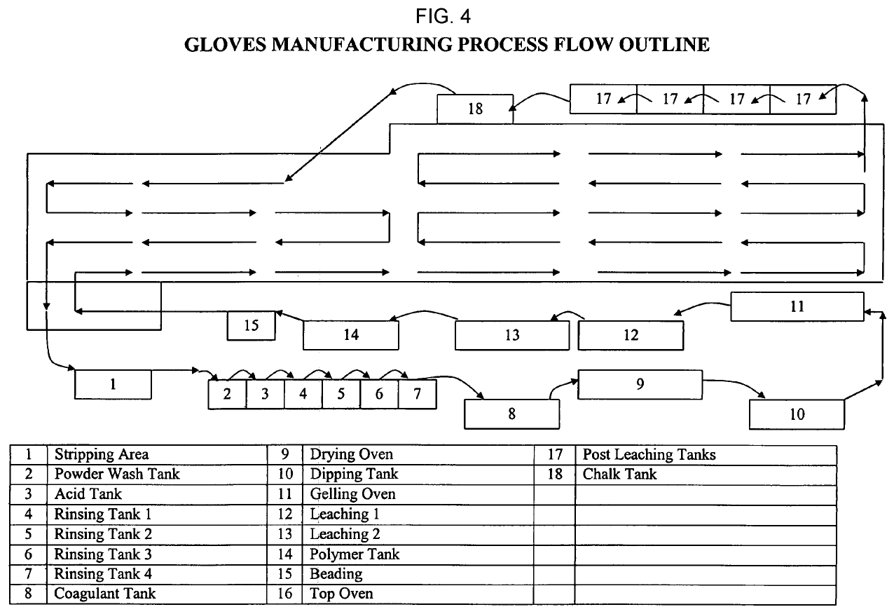

Referring to FIGS. 1 and 2, the present invention is a process for manufacturing a thin-walled disposable glove 1 as shown in FIG. 1, which has been manufactured in such a way that the donning surface 4 is uniformly coated with a layer of dehydrated Lano-E composite 2. The Lano-E composite 2 is comprised of a coating of Lanolin and Vitamin E that is adequate to provide a well-balanced, non-greasy optimum smoothness without affecting the durability and flexibility of the bulk materials that make the glove 1. The glove 1 can be made of natural rubber latex or synthetic materials such as acrylonitrile butadiene, polyurethane, polychloroprene and polyvinyl chloride. In one preferred embodiment, the material is natural rubber latex. FIG. 3 details the steps in the special process for applying the Lano-E composite 2 coating. The glove 1 has an interior 3 with a donning surface 4 and an exterior surface 5. The gloves 1 are initially manufactured by a dipping process, which is a well known method of manufacturing within the industry. FIG. 4, “Gloves Manufacturing Process Flow Outline” briefly details the basic steps in such a dipping process. Once the glove manufacturing process is complete the process for application of mixture of Lanolin and Vitamin E is begun.

The mixture of Lanolin and Vitamin E or rather Lano-E composite 2 is formulated in such a way that the composition is optimized and adequate to provide a well-balanced, non-greasy optimum smoothness without affecting the durability and flexibility of the bulk materials of glove. The Lano-E composite 2 is evenly applied onto the donning surface 4 of glove. In one preferred embodiment the thickness of the dehydrated coat of Lano-E composite 2 is about 0.01 mm. A range of about 0.005 mm to about 0.02 mm coating of Lano-E 2 has been found to provide a well balanced, non-greasy optimum smoothness without affecting the durability and flexibility.

The application of the mixture of Lanolin and Vitamin E to the glove involves treating the glove 1 by a series of washing processes that remove the powder, water-soluble impurities and microbes. The incoming powdered glove 1 is first water rinsed (R-1) to remove excess powder from the exterior surface of the glove. A glove 1 in its normal orientation is then inverted (11) inside-out so that the donning surface 4 becomes the outside external portion of the glove and the normally exterior surface 5 is temporarily the internal portion of the glove. The gloves 1 are again rinsed (R-2) with water to remove excess powder from primarily the donning surface 4 of the glove. The glove 1 is exposed to series of controlled chlorination washing processes.

The glove 1 is exposed to chlorine gas (C1) in an enclosed chamber using water as the aqueous medium to facilitate the reaction to take place. The intensity of chlorination is carefully controlled to optimize the degree of treatment on both the donning surface 4 and the exterior surface 5 of the glove without overly exposing the surfaces to the chlorine gas. The chlorine gas is produced from reactions between Sodium Hypochlorite and Hydrochloric acid. The chlorine is at a concentration of about 850 ppm in water, with a range of between about 700 ppm and 1500 ppm being satisfactory. Over exposure to the chlorine gas can be very damaging to the physical properties of the finished product.

Following the chlorination washing process the glove 1 is water rinsed two more time in rinse (R-3) and rinse (R-4) to remove the chlorine residue. On completion of the chlorine gas washing and the water rinsing cycles (R-3 and R-4), the glove is then treated with Lano-E composite 2 before heating (D1 and D2) the glove 1 to complete dryness. A visual assessment is performed upon completion of the drying process.

Lano-E composite 2 is first prepared in a bulk quantity of concentration comprising of Pure Lanolin solution and Vitamin E in the form of alpha-tocopheryl acetate. The mixture is blended homogeneously with warm water at 45 degree Celsius to form an emulsified mixture. The water temperature can vary from between about 35 degree Celsius to about 55 degree Celsius. To apply the composite 2 onto the donning surface 4, the emulsified mixture can be sprayed onto the surface of the glove 1. A pre-defined quantity of the emulsified mixture shall be determined and followed for the treatment.

About 15,000 glove pieces can be coated at one time by spraying the emulsified mixture. The treated glove 1 will then be dried through a series of carefully controlled drying procedures in a tumbling dryer. The tumbling mechanism is preferred because the system ensures each and every piece of the gloves 1 is kept in continuous motion while they are being dried. Such drying process is conducive for the formation of a thin and uniform coating of the Lano-E composite 2 on the glove 1. The coating may vary, with a preferred range of from about 0.005 millimeters to about 0.02 millimeters.

The drying process may vary in length of time and temperature. In one effective variation the water or other liquid carrying medium of the composite is slowly evaporated in two stages:

| | | |
|---|---|---|
| Stage | Drying temperature (° C.) | Drying time (min) |

Synthetic Gloves

| | | |
|---|---|---|
| First Drying | 50 to 90 | 20 to 180 |
| Second Drying | 60 to 90 | 30 to 60 |

Natural Rubber Gloves

| | | |
|---|---|---|
| First Drying | 40 to 70 | 20 to 120 |
| Second Drying | 40 to 60 | 50 to 120 |

Upon completion of the first drying process (D1), the glove 1 is inverted again so that the glove 1 returns to its normal orientation where the donning surface 4 faces inside. The gloves 1 are subsequently subjected to the Second Drying (D2) process, if applicable, until complete dryness is achieved.

Excessive heating can bring about quality problems in which the glove 1 may turn brownish in color and develop a pungent smell on completion of the drying process.

Besides the built-in automated temperature regulating system, each drying machine is installed with an over-heating alarm system as an additional quality control feature to safeguard the quality of the drying process. Should the drying temperature exceed the required setting, a warning signal will be triggered and the heating mechanism will be deactivated immediately. The faulty unit will not be used until it is repaired.

The gloves 1 are inverted in between the First drying (D1) and the Second Drying (D2). This glove 1 inversion is so that the glove 1 returns to its normal orientation, where the donning surface 4 faces inside and is the internal portion of the glove. The inversion processes are performed manually with the aid of air nozzles driven by vortex blowers.

At the completion of the process the donning surface 4 is uniformly coated with a layer of dehydrated Lano-E composite 2, which is comprised of measured parts of Lanolin and Vitamin E that is adequate to provide a well-balanced, non-greasy optimum smoothness for the wearer of the glove 1.

Several factors in the process contribute to the ultimate satisfactory coating of dehydrated Lano-E composite 2, including, but not limited to, the ratio of Lanolin and Vitamin E, the quantity of Lanolin and Vitamin E applied to the donning surface 4, the chlorine wash, the component ratios in the emulsified mixture with the water, and the staged drying process.

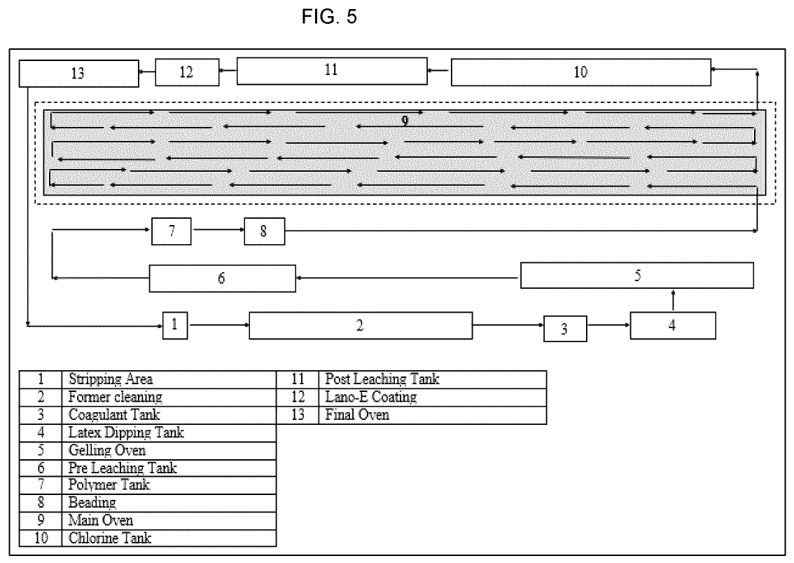

In a preferred embodiment, the first treatment process explained above is used for online powdered glove that requires further offline treatment. Powder free glove that does not require offline treatment will undergo this newly added Lano-E online treatment process. FIG. 5, “Lano-E Online Treatment Process” shows the online application of Lano-E coating during the manufacturing process of powder free gloves.

Based on FIG. 5, the application of the mixture of Lanolin and Vitamin E is carried out by spraying the mixture onto the donning surface 3 of glove, dried in a controlled heating condition before reaching the stripping area. During the manufacturing process of powder free glove, the donning surface of glove is exposed to the outside. Lano-E composite 2 is prepared in a tank and connected to spraying nozzles. The nozzles are fixed on top of the slurry tank at both sides of lines whereby they spray the composite from top while hand molds move horizontally. The hand molds rotate while moving, thus leaving a uniform coating of Lano-E composite 2 on the donning surface 3 of glove. Then, the glove goes through a drying zone with controlled heating condition whereby the temperature is set within a range of 50 to 60 degree Celsius. The glove inverts out to its normal orientation when stripped out from hand mold using automatic robotic grippers.

Although the present invention has been described in considerable detail with regard to the preferred versions thereof, other versions are possible. Therefore, the appended claims should not be limited to the descriptions of the preferred versions contained herein.

1. A method of manufacturing a hand health care disposable glove comprising:
a) forming a disposable online powdered glove from natural rubber latex or other synthetic materials selected from the group consisting of acrylonitrile butadiene, polyurethane, polychloroprene or polyvinyl chloride through a dipping process, wherein in normal orientation the glove having an interior with a donning surface and having an exterior surface;
b) inverting said gloves inside out, whereby the donning surface is exposed;
c) subjecting said glove through a chlorination washing process;
d) covering the donning surface of said glove with a coating of an emulsified mixture of Lanolin and Vitamin E (Lano-E composite) using a liquid medium, wherein the Lano-E composite is formed by blending Lanolin and Vitamin E homogeneously with water between about 35 degrees Celsius and about 55 degrees in Celsius, wherein the glove is sprayed with the emulsified mixture;
e) evaporating the liquid medium from the emulsified mixture in a heating condition, whereby a dried coating of the Lano-E composite is formed on the donning surface of said glove, wherein evaporating takes about 20 to 180 minutes at 50 to 90 degrees in Celsius for synthetic gloves, and about 20 to 120 minutes at 40 to 70 degrees in Celsius for natural rubber gloves;
f) inverting said glove such that the donning surface is taking inside; and
g) second drying for about 30 to 60 minutes at 60 to 90 degrees in Celsius for synthetic gloves, and about 50 to 120 minutes at 40 to 60 degrees in Celsius for natural rubber gloves if applicable.

2. The method of claim 1, wherein the liquid medium comprises water.

3. The method of claim 1, wherein said covering comprises purified lanolin enriched with Vitamin E.

4. The method of claim 3, wherein said covering comprises spraying said glove with said emulsified mixtures of Lanolin and Vitamin E in said liquid medium.

5. The method of claim 1, further comprising pre-treating the donning surface of said glove with a series of two or more chlorination washing processes prior to covering the donning surface with said emulsified mixture of Lanolin and Vitamin E using said liquid medium.

6. The method of claim 1, wherein the Vitamin E is in the form of alpha-tocopheryl acetate.

7. The method of claim 1, wherein the Lano-E composite is formed by blending Lanolin and Vitamin E homogeneously with about 45 degrees Celsius water.

8. The method of claim 1, wherein a thickness of the coating of the Lano-E composite when dehydrated is between about 0.005 mm and about 0.02 mm.

9. The method of claim 8, wherein a thickness of the coating of the Lano-E composite when dehydrated is about 0.01 mm.

10. The method of claim 1, further comprising a first rinsing of the glove, prior to the inversion of said glove, whereby the rinsing removes powder residue.

11. The method of claim 10, further comprising a second rinsing of the glove, following the inversion of said glove, whereby the rinsing removes powder residue.

12. The method of claim 11, further comprising a third rinsing of the glove, following the chlorination washing process, whereby the rinsing removes chlorination residue.

13. The method of claim 12, above further comprising a fourth rinsing of the glove, following the chlorination washing process, whereby the rinsing removes chlorination residue.

14. A method of manufacturing a hand health care disposable glove comprising:
a) forming a disposable online powder free glove from natural rubber latex or other synthetic materials selected from the group consisting of acrylonitrile butadiene, polyurethane, polychloroprene or polyvinyl chloride through a dipping process, wherein the donning surface is exposed during the manufacturing process of the powder free glove;
b) covering the donning surface of said glove with a coating of an emulsified mixture of Lanolin and Vitamin E (Lano-E composite) using a liquid medium, wherein the Lano-E composite is formed by blending Lanolin and Vitamin E homogeneously with water between about 35 degrees Celsius and about 55 degrees in Celsius, wherein covering the donning surface of said glove with a coating of the emulsified mixture of Lanolin and Vitamin E (Lano-E composite) includes spraying on a hand mold of said glove while the hand mold moves horizontally on a line and rotates while moving horizontally;
c) evaporating the liquid medium from the emulsified mixture in a controlled heating condition, whereby a dried coating of the Lano-E composite is formed on the donning surface of said glove, wherein the temperature is set within a range of 50 to 60 degrees Celsius; and
d) inverting said glove at stripping area such that the donning surface is taking inside.

15. The method of claim 14, wherein the liquid medium comprises water, wherein inverting said glove at the stripping area such that the donning surface is taking inside includes inverting said glove out to its normal orientation when stripped out from the hand mold using automatic robotic grippers.

16. The method of claim 14, wherein said covering comprises purified lanolin enriched with Vitamin E.

17. The method of claim 16, wherein said covering comprises spraying said glove with said emulsified mixtures of Lanolin and Vitamin E in said liquid medium.

18. The method of claim 14, wherein the Vitamin E is in the form of alpha-tocopheryl acetate.

19. The method of claim 14, wherein a thickness of the coating of the Lano-E composite when dehydrated is between about 0.005 mm and about 0.02 mm.

20. The method of claim 14, wherein the mixture of Lanolin and Vitamin E (Lano-E composite) is prepared in a tank and connected to spraying nozzles, wherein the nozzles are fixed on top of the tank at both sides of the line, wherein the spraying nozzles spray the composite from the top while the hand mold of said glove moves horizontally.
